(19) United States
(12) Patent Application Publication
(10) Pub. No.: US 2017/0143445 A1
(43) Pub. Date: May 25, 2017

(54) METHOD AND APPARATUS FOR OPERATING A DENTAL DIAGNOSTIC IMAGE GENERATION SYSTEM

(71) Applicant: DENTSPLY SIRONA Inc., York, PA (US)

(72) Inventor: Ciamak ABKAI, Heddesheim (DE)

(73) Assignee: Sirona Dental Systems GmbH, Bensheim (DE)

(21) Appl. No.: 15/355,170

(22) Filed: Nov. 18, 2016

(30) Foreign Application Priority Data
Nov. 19, 2015 (DE) 102015222821.5

(51) Int. Cl. A61B 90/00; A61B 5/00; A61F 5/56; A61B 6/14; A61B 6/00; A61B 5/055; A61B 6/03

(52) U.S. Cl. CPC A61B 90/39; A61B 5/055; A61B 5/4542; A61B 5/4836; A61B 6/032; A61B 6/145; A61B 6/5247; A61F 5/566; A61B 2090/3912; A61B 2090/3937; A61B 2090/3954; A61B 2090/3966; A61B 2090/3995

(57) ABSTRACT

Disclosed is a method for operating a dental diagnostic image generation system of at least two modalities for generating three-dimensional image data by means of X-rays and at least one further beam of a different modality, wherein at least two three-dimensional image data of different modalities are generated and the at least two three-dimensional image data are merged by means of a multimodal registration, it is in particular provided that the image data generation of the at least two three-dimensional image data at an object to be imaged is carried out with markers which overlap during the at least two irradiations of different modalities, that the image data generation of at least one modality is carried out with a capture area limited in accordance with the markers, and that the at least two three-dimensional image data is spatially associated by means of multimodal registration on the basis of the markers captured in the three-dimensional image data.

### TECHNICAL FIELD

The present disclosure relates to an image generation or image display system suitable for techniques used in dental medicine or dental diagnostics for temporomandibular joint diagnosis and temporomandibular joint therapy, in which at least two three-dimensional image data sets are multimodally merged or combined, wherein at least one of the at least two three-dimensional image data sets is generated tomographically, and in particular relates to a method for operating such an image generation system used in temporomandibular joint diagnosis and temporomandibular joint therapy. The present disclosure furthermore relates to a computer program, to a computer-readable medium for storing the computer program, and to an apparatus, as well as an image generation and image display system, by means of which the method to which this disclosure relates can be implemented.

### BACKGROUND

Modern image generation systems, particularly those used in dental medicine, produce image data or volume data which represent the object to be imaged in its three-dimensionality (3D) and which must be prepared and displayed for the user or viewer. In the meantime, it has now become possible to access 3D image data of the object to be treated which were obtained preoperatively or intraoperatively, for example, image data of a human jaw or tooth, in order to make a diagnosis or draw up plans before a medical procedure.

The known method of three-dimensional or spatial dental or digital volume tomography (DVT) is here already employed in dentistry, said method using a conical bundle of rays and representing a three-dimensional imaging tomographic method using X-rays in which cross-sectional images are generated. As in digital radiography, so too in DVT, an X-ray tube and an opposite image sensor or detector, which has, for example, a layer of scintillator sensitive to X-rays, rotate around a lying, sitting or standing patient. The X-ray tube, which usually rotates 180 to 360 degrees with a fan angle, emits a conical X-ray beam which in most cases is pulsed.

In addition, dental medicine or dental diagnostics are increasingly using even optical scanning methods, for example, by means of so-called CEREC devices or systems, in which CAD/CAM methods are applied to reconstruct dental restorations. This enables dentists themselves to design patient-specific ceramic restorations with computer support, efficiently and expeditiously, by working directly on a treatment unit in a single treatment appointment, and making and possibly even fitting the patient with the said restorations. Here an optical impression of the anatomical object to be treated, such as a tooth stump intended for an inlay or a crown, is generated by means of an intraoral camera and a three-dimensional model is calculated with computer support from the optical image data obtained.

During generation of the optical image data, a corresponding counterbite can even be included in the calculations. With the aid of a combined copying/grinding process, the calculated restoration (an inlay, for example) is milled out of a ceramic block by a three-axis grinding machine with the appropriate grinding tools.

Alternatively, a video camera described in DE 42 26 990 A1 can be used for recording or generating optical 3D image data, by means of which objects in the oral cavity of a patient can be observed and recorded and from the image data thus acquired the corresponding optical 3D image data can be calculated using a computer.

Following the creation of said 3D radiographic images and optical 3D images, it is necessary to bring these image data into spatial alignment for a subsequent diagnosis or another dental treatment process - in other words, to carry out a so-called ‘multimodal registration’ or ‘3D-3D registration’. A process of this kind, for combining or merging 3D image data captured by said optical scanning with 3D image data sets otherwise generated, is disclosed by WO 2009/140582 A2. Here multiple polygons are linked or stitched together to create a polygon-mesh-based electronic model of the object to be examined or imaged.

In addition, DE 10 2007 001 684 A1 describes a method for the image registration of volume and surface data by means of which a precise and automated registration of an optical recording with an X-ray of a patient is made possible. With this method, a said registration, in which the optical recording can be made to coincide spatially with the tomographic recording, can be carried out without using any external reference bodies, physical models, such as plaster models, or mechanical devices. Registration is here largely automated and can be carried out in the relatively short time of about 15-30 seconds. In particular, a transform function is used to bring a distinctive volume structure, extracted from the volume data and formed, for example, by the edges of the object, into the closest possible alignment with the corresponding structure in the surface data, wherein a measure for the quality of the congruence is defined and wherein, in iterative steps optimizing the said quality measure, the extracted structure is adjusted to the surface structure apparent in the surface data. As a result, the coordinates of the optical recording are brought into alignment with the coordinates of the X-ray by iteration.

Furthermore, a prototype of the registration of a radiographic data set of the jaw and of the surface data set of a corresponding plaster model is known from the publication ‘Fusion of computed tomography data and optical 3D images of the dentition for streak artifact correction in the simulation of orthognathic surgery’, Nkenke E., Zachow, S. et al., (published in Dentomaxillofacial Radiology (2004), 33, 226-232). Here, first of all, the visible surface—in other words, the surface of the teeth and mucous membrane—is extracted from the X-ray of the plaster model before being registered in the next step with the surface from the optical recording with the aid of an ICP algorithm (‘iterative closest point’). In practice, however, this method can hardly be used since extraction of the surface from the radiographic data set of a real patient record is imprecise and the requirements for a precise registration of the surfaces are therefore not met.

Moreover, the use of reference bodies (markers) for the registration concerned here is known. However, due to the associated issues of attachment and the discomfort for the patient, markers are not used unless there is no simpler way. Accordingly, U.S. Pat. No. 5,842,858 discloses a method in which the patient wears a template with markers when the X-ray is taken, said template then being placed on a model to which a sensor for 3D-positional capture is attached. Once the positional relationship between the sensor and the markers has been determined, the template can be removed and an optical recording made. Here the 3D sensor makes registration possible in relation to the patient recording.

A so-called SICAT function has recently been added to the image generation systems concerned here, said function enabling, for example, the anatomically accurate representation of the movement of a lower jaw within a 3D volume, wherein the movement tracks of the temporomandibular joint can be visualized and reproduced for any point by means of an anatomically correct trajectory.

An apparatus and a method for measuring jaw movement is disclosed by DE 10339241 A1. The apparatus has a pair of fixed markers attached to both sides of the face of a patient, as well as a pair of movable markers which are so arranged that they face the fixed markers at a distance and move integrally with the movement of the lower jaw. In addition, four cameras are arranged which, during movement of the lower jaw, record the three-dimensional movement of the movable markers relative to the fixed markers. This apparatus makes possible a precise mapping of the center of rotation of a movement of the lower jaw, as well as the corresponding spatial trajectory of the lower jaw.

### SUMMARY

Disadvantageous in this known method is that to make a said multimodal registration of the 3D image data concerned here, it is necessary also to map the anatomical area surrounding the object to be imaged, radiographically or tomographically covering a large area or large volume, in other words, for example, by an irradiation technique.

The present disclosure is based on the idea relating to the case of a here pertinent image generation system and a method of operating it, in which a first three-dimensional image data set of volume data is provided, which represents an area of the jaw of a patient which in particular covers at least one temporomandibular joint and which is recorded by a transilluminating imaging tomographic technique of a first modality, and in which at least a second three-dimensional image data set of surface data is also provided, which at least partially represents the same area of the jaw of the patient and which is recorded by a technique of a second modality for recording visible surfaces, said idea providing for the image data generation of the at least two three-dimensional image data sets being carried out at the object to be imaged with markers recorded during the at least two recordings of different modalities, wherein image data generation at least by means of the tomographic first recording is carried out with a recording area demarcated in accordance with the markers, and the at least two three-dimensional image data sets are spatially associated with regard to the temporomandibular joint by means of the multimodal registration on the basis of the markers recorded in the three-dimensional image data of both modalities.

Accordingly, the first image data set is preferably produced radiographically or tomographically, in particular by a said digital volume tomography (DVT), wherein according to the present disclosure the radiographic or tomographic (capture) volume is limited spatially in such a way that if at all possible it represents only the anatomical or medical situation of the particular dental object to be imaged, this including at least one temporomandibular joint, for example, of a temporomandibular joint. This limitation of the said volume serves in particular to limit or minimize the radiation dose involved in image generation or the radiation exposure for the patient or operating personnel and also to limit the duration of irradiation, thereby securing cost benefits.

It should be emphasized that a here pertinent image generation system can also be a distributed system, in which the said image data are created by various image generation devices and/or at different times and do not yield information relevant to treatment until these image data sets are overlaid. In this way, computer tomographic (CT) or cone-beam (CB) radiographic recordings of the jaw of a patient which contain detailed anatomical information can be prepared for use in planning a dental medical procedure, such as a dental implant. On the other hand, in the planning and fabrication of dental prosthetic restorations, three-dimensional surface images can be acquired directly from the jaw or an impression of the jaw by means of an optical recording unit, for example, a CEREC device or system of the present applicant. In contrast to tomographic recordings, these surface data contain information about the course of the visible surface of the jaw in question, in particular the surface of the teeth and the mucous membrane.

### DETAILED DESCRIPTION

The method according to the present disclosure, which is in particular suitable for temporomandibular joint diagnosis and temporomandibular joint therapy, can arrange for relative movements (or the corresponding ‘condylography data’) of the two jaws to be recorded with the aid of a signal transmitter and a signal receiver attached rigidly to the upper jaw and lower jaw, and by means of the condylographic data so obtained determine the trajectory of an imaginary hinge axis of the two temporomandibular joints during chewing, this being carried out, for example, by an attending physician. The said signal transmitter and corresponding signal transmitter may be ultrasonic devices. On the basis of the trajectory thus determined, which corresponds to an imaginary hinge axis, the attending physician can, with the aid of the axis track of the trajectory, make a diagnosis of, for example, pathologies of the temporomandibular joints.

The method according to the present disclosure concerns in particular the image generation of first 3D image data generated, for example, by means of a said DVT or MRT process or system, and of at least second 3D image data optically captured or scanned by, for example, a said CEREC device or system, wherein the image data generated are precisely assigned to each other spatially or these data are to be brought into spatial alignment (so-called ‘multimodal registration’). By means of the multimodal registration, the two 3D image data sets can be merged accordingly for the purpose of an integrated representation (so-called ‘data fusion’).

To enable multimodal registration of the image data already generated, according to the present disclosure natural or anatomical markers are defined in the patient during generation of the first and second 3D image data sets, preferably within the oral cavity of the patient, or a special marking device, such as a template, is fitted which, due to its shape or a suitable arrangement of preferably not only optical but also radiographically measurable or visible markers or structures, allows a said subsequent assignment or merging of the first and second 3D image data sets.

Such a template can be produced at low cost and in the case of a here pertinent image generation process is simple to use. The template can have a configuration or arrangement of markers or structures which is visible in the at least two modalities, by means of which multimodal registration of the three-dimensional image data generated is subsequently easy to carry out.

For the marker device or template to allow a precise multimodal registration or 3D-3D registration it must be arranged with the best fit possible in that part of the patient's oral cavity which is relevant to image generation, in other words, mechanically and/or frictionally interlocked, for example, in order to prevent it shifting or changing position during both image-acquisition operations. For this reason, a template is preferred which is temporarily fixed between individual teeth or rows of teeth similar to the braces or the like used in orthodontic treatment, or placed temporarily on entire rows of teeth in a similar way to a therapy brace.

The said optical generation of first image data and also the said radiographic generation of the second image data must therefore both cover or capture at least one relevant part of the template with regard to the said marker or structure arrangement. Not only during optical capture but also during radiographic capture the template's configuration, structure arrangement or markers must here be detectable or visible in the 3D image data generated in each case so that partial information or information overlapping both sets of image data from the template are visible in both image data sets. In this way, a multimodal registration of said different modalities (for example, optical and radiographic capture) is possible which is a considerable improvement on using optically captured tooth surfaces.

The multimodal registration can be carried out by, for example, assigning X-ray markers of the DVT to artificial optical markers captured by optical scanning. As an alternative or addition to the said artificial markers, natural or anatomy-based markers, for example, the surfaces of optically captured teeth, can be used.

The said structural arrangement of the template can involve at least three hole-like or punctiform perforations arranged at a distance from each other in order to make the here pertinent multimodal registration possible by means of the ICP algorithm described in even more detail below. US 2009/0316966 A1 discloses a similar method for registering first 3D image data of an anatomical model (dental model) with second 3D image data obtained by a 3D image generation process, wherein the said ICP algorithm is employed. In this way overlapping areas or the corresponding image data can be eliminated from one of the two image data sets. However, this concerns a different application scenario.

By means of a template according to the present disclosure it is in particular possible to limit the radiographic area, for example, the captured DVT volume, considerably as compared with prior art, and thus reduce the required radiation dose, since advantageously the merging of the 3D image data no longer has to be done on the basis of further anatomical structures arranged around the object to be imaged. In this way, radiation exposure for the patient and operating personnel is minimized on the one hand and the costs of radiographic generation of the second 3D image data are considerably reduced on the other.

When the said template is used, there is accordingly no need for high-volume radiographically generated first 3D image data or volume information as created in prior art by, for example, a high-volume DVT, such as by means of a known ‘Galileos’ image generation system. Accordingly, with the method according to the present disclosure for a treatment or diagnosis in the anterior tooth region of a patient, or in the case of a diagnosis which is only concerned with the position of the teeth in the context of a temporomandibular joint location, it is no longer necessary to capture the entire jaw or dental arch radiographically or X-ray tomographically.

On the basis of the mutually assigned or merged 3D image data produced according to the present disclosure, a multimodal functional analysis of, for example, the temporomandibular joints of the patient can be performed. In a functional treatment or diagnosis of this kind, being able to design or fabricate an appropriate brace means that considerable importance is attached firstly to the position or spatial location of the temporomandibular joint socket and temporomandibular bones during movement or dynamics and secondly to the corresponding mutual orientation or positioning of the teeth in the upper jaw and lower jaw.

For the fabrication of a said dental brace, firstly, the relatively high image resolution, for example, of a said CEREC device or system, is exploited in the generation of the first 3D image data. Secondly, the second 3D image data captured radiographically, for example, by means of the said DVT method or system, serve to capture the coupling of the dynamics or movement to the lower jaw. This is because the area of the joint socket or joint bones can be captured by a here pertinent optical image generation system, for example, by a said CEREC device or system, not only from the inside but also from the outside (with respect to the oral cavity of the patient). This area can only be captured tomographically, for example, by a CT or MR process.

The method according to the present disclosure for operating a here pertinent image generation system, therefore, particularly provides for image data generation of the at least two three-dimensional image data being carried out at an object to be imaged with markers captured in the at least two recordings or exposures of different modalities, for image data generation of at least one modality being carried out with a capture area or recording area limited in accordance with the markers, and for the at least two three-dimensional image data to be spatially associated by means of multimodal registration on the basis of the markers captured in the three-dimensional image data. The said markers supply reference data, so to speak, for carrying out the said multimodal registration. The said tomographic recordings can be acquired by magnetic resonance tomography (MRT) or by radiography.

The two modalities are preferably provided by a said DVT method and a said CEREC method.

As an alternative to using a said template, it can be envisaged that multimodal registration is carried out by associating natural or anatomy-based markers. Here it is possible to manage without a said template but it can be harder to find suitable anatomical markers which are detectable or visible in the image data generated in the at least two modalities.

The method according to the present disclosure is applicable in particular with a here pertinent dental medical or dental diagnostic image generation system equipped with a said SICAT function, and thereby makes possible a functional analysis of moving anatomical objects even in the generation of here pertinent 3D image data in an only relatively small field of view (FoV). In this way, a functional analysis of temporomandibular joints can be carried out by the operation according to the present disclosure of a multimodal image generation system, wherein in particular the radiographic images involved are generated with a radiation dose which is relatively low in comparison with prior art.

When a so-called ‘Cerec Omnicam’, described below, is used, the result of a 3D-3D registration according to the present disclosure can be further improved or stabilized with additional color information, since additionally processable material information for 3D-3D registration is made available by means of precise optical 3D images including color information.

The computer program according to the present disclosure is set up to perform each step of the process, in particular when it runs on a computing device or a controller. It allows the method according to the present disclosure to be implemented on an electronic control unit without the need to make structural modifications to said unit. The machine-readable data medium on which the computer program according to the disclosure is stored is provided for this. By downloading the computer program according to the present disclosure onto an electronic control unit, the electronic control unit according to the present disclosure is obtained which is set up to control a here pertinent image generation system by means of the method according to the disclosure.

The present disclosure further relates to a dental-medical image generation system which is set up to be controlled by a method according to the disclosure, wherein the said advantages emerge.

Further advantages and embodiments of the present disclosure will be apparent from the description and the accompanying drawings.

It is understood that the features mentioned above and those yet to be explained can be applied without departing from the scope of the present disclosure, not only in the respective combinations indicated but also in other combinations or on their own.

### BRIEF DESCRIPTION OF THE DRAWINGS

FIG. 1 shows a schematic representation of a lower jaw layer in the axial direction with a template in accordance with the disclosure being used.

FIG. 2 shows an embodiment of the method according to the disclosure with the aid of a flow chart.

FIGS. 3a - 3d show in schematic form a calculation example of a multimodal registration according to the disclosure.

FIG. 4 shows an embodiment of a possible image merging or image data fusion using optical and radiographic markers.

FIG. 5 shows an embodiment of a template for possible use in an image merging or image data fusion according to FIG. 4.

### EXEMPLARY EMBODIMENTS

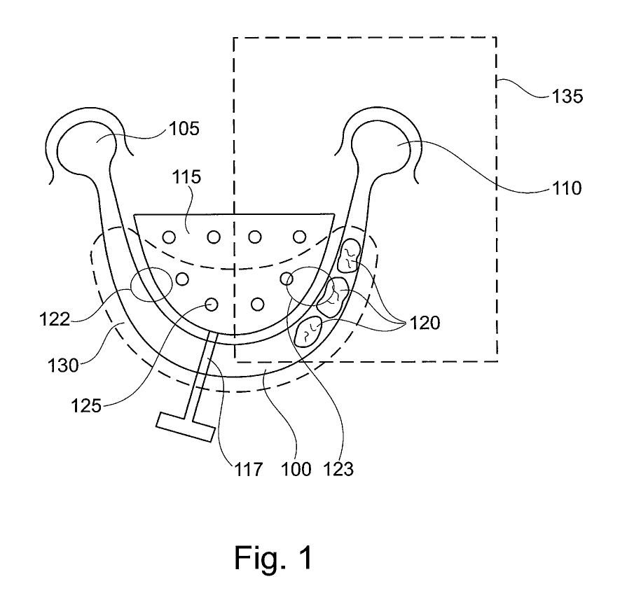

FIG. 1 shows a schematic representation of an axial lower jaw layer 100 with the two temporomandibular joints 105, 110 of a patient for the purpose of explaining an image data fusion made possible according to the present disclosure by combining or overlaying in this case three different modalities, namely optical CEREC acquisition or generation, radiographic DVT acquisition or generation, and also the template. Here the lower jaw 100 can during optical and radiographic image data capture be moved around the axis of rotation formed by the temporomandibular joints 105, 110 in order to be able to also carry out a specified functional analysis of the temporomandibular joints 105, 110.

During the entire image data capture process of the various modalities, a template 115 preferably provided with a handle 117 and shown in FIG. 1 is clamped between the rows of teeth 120 of the lower jaw 100, for example, in the two areas 122, 123, with precise fit or seating, that is, mechanically and/or frictionally interlocked. In the present exemplary embodiment, the template 115 has eight perforated holes or openings 125 which serve as so-called markers for the subsequent multimodal or 3D-3D registration.

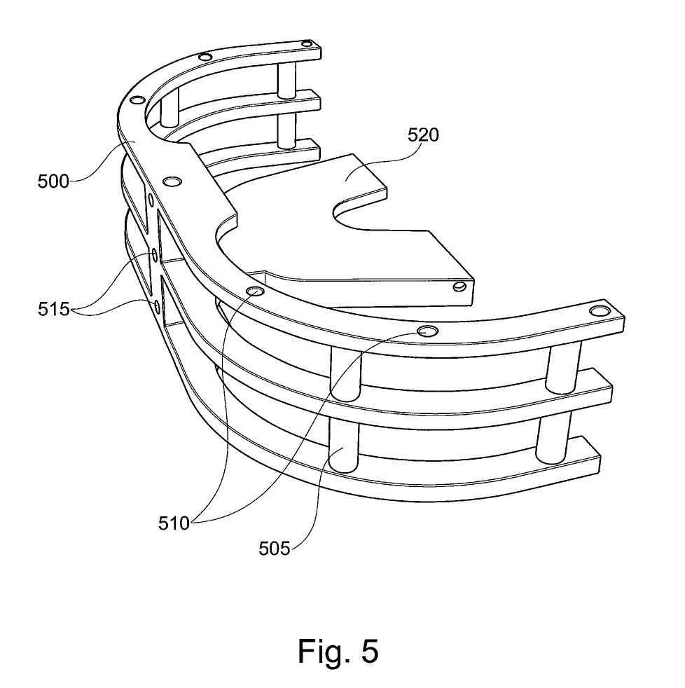

The template 115 described can be made of a ceramic material or a metal, such as stainless steel. The optical markers mentioned can be balls, cylinders or pins, or be based on particular textures. The markers can also be color-coded and, for example, take the form of blue balls or balls which emit light in the ultraviolet range. A further exemplary embodiment of the template is shown in FIG. 5 and is described below.

The template 115 is so arranged in the oral cavity of the patient that during image data capture the scan area 130 of the optical CEREC recording and also the radiographic DVT volume 135 overlap, that is, scan or transilluminate, not only the relevant three teeth 120 to be imaged, but also at least a relevant part of the template 115. At least three holes 125 in the template 115 which are required for a spatial 3D-3D registration serve as a relevant part, wherein the relevant part preferably represents the intersection of the CEREC scan area 130 and the DVT volume 135, wherein in the present exemplary embodiment even the bottom four of the total of eight holes 125 are covered by the data-collecting optical rays 130 and the X-rays.

Should there be an overlap between the optical CEREC recording and the tomographically or radiographically recorded DVT recording, these two recordings must be related spatially by means of a transformation from known marker data. Here X-ray markers, such as ceramic balls, can in a known manner be used for the tomographic area and, in turn, optically effective markers, for example, once again balls or cylinders with a particular color or surface texture can be used in a known manner for the optical area. The spatial relationship between these markers is determined by a transformation matrix as described below, namely on the basis of, for example, transformation data supplied by the manufacturer for that particular template.

Since not only the optical CEREC recording but also, in particular, the radiographic DVT recording only need to be created in the said relevant area of the template 115, the exposure of patient and operating personnel to radiation is considerably reduced in comparison with prior art.

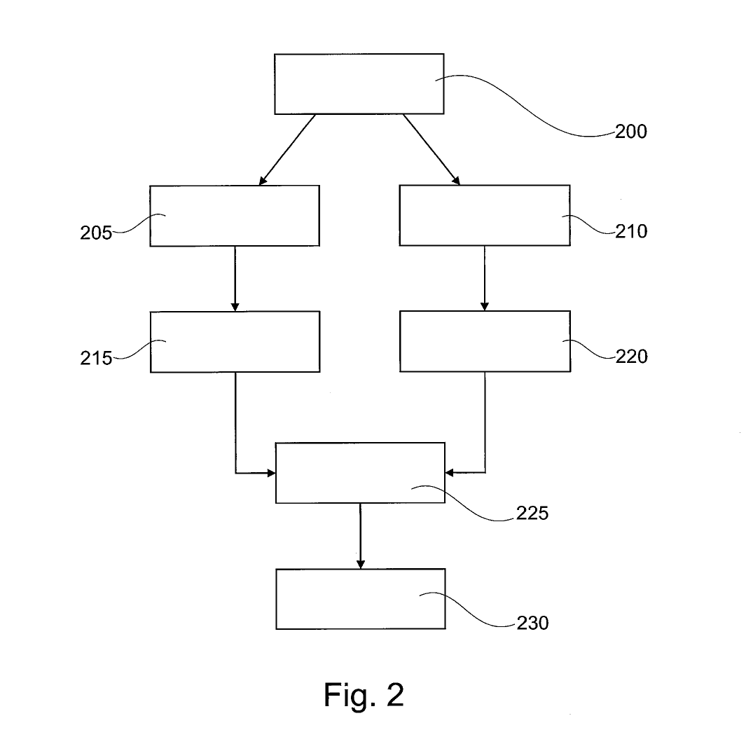

In the flow chart shown in FIG. 2 for carrying out an image capture according to the present disclosure and the subsequent 3D-3D registration, first of all a template according to the disclosure is installed 200 at a specified suitable location in the oral cavity of the patient being examined. It should, however, be emphasized that the method according to the disclosure can also be applied without using a template of this kind, wherein suitable anatomical features, such as the surfaces of the three teeth shown, can serve as the basis during registration instead of the template.

Once the template has been installed 200, in the present exemplary embodiment, optical 3D image data are generated 205 by the said CEREC method in the scan area shown. Simultaneously, previously or subsequently, radiographic 3D image data are generated 210 by the said DVT method, wherein the DVT volume used here overlaps the said CEREC scan area at least in the area of the anatomical objects to be examined, that is, in the present case the three teeth 120 shown in FIG. 1 and also the said relevant marker areas of the template. In the said 3D image data acquisition using CEREC and DVT the image data are in all cases captured together with 3D coordinates.

In the optical and radiographic 3D image data thereby generated, in each case the said markers (for example, three said holes) of the template are identified 215, 220 and on the basis of the identified markers a 3D-3D registration of the optical and radiographic image data is carried out 225 with the aid of the ICP algorithm described below. 3D-3D registration yields optical and radiographic image data 230 which fit together anatomically correctly and can therefore be simultaneously displayed or superimposed, for example, on a monitor.

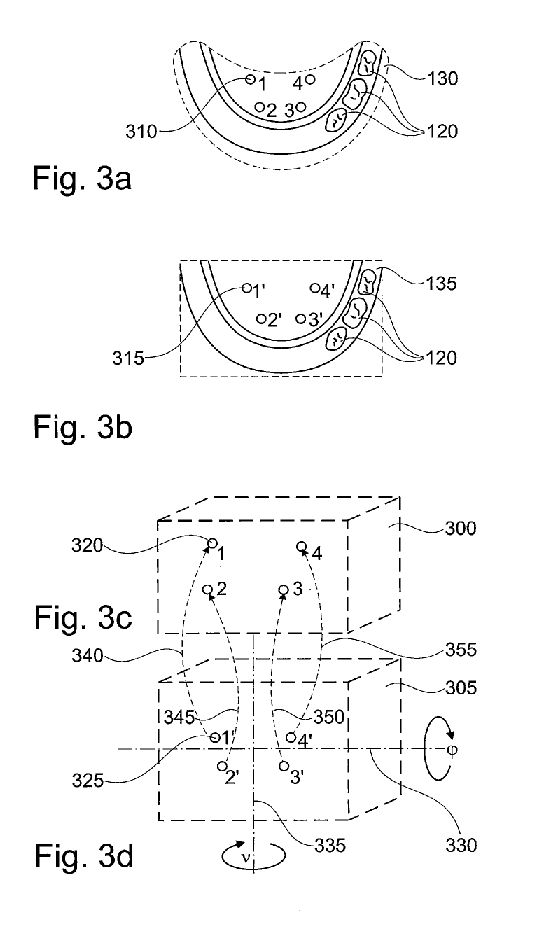

For 3D-3D registration of the first and second 3D image data sets, the known ‘Iterative Closest Point’ (ICP) algorithm, shown in schematic form in FIGS. 3a- 3d, is used, by means of which the point clouds 310, 315 contained in the 3D image data and shown here in schematic form can be mutually adjusted or brought into spatial alignment. As can be seen in FIGS. 3a and 3b, the DVT recording made in the DVT volume 135 is tilted relative to the optical recording made in the CEREC scan area 130. This results, for example, from the three teeth 120 covered by the recordings which in FIG. 3a were basically recorded axially from above, whereas these teeth 120 according to FIG. 3b were captured more from the side in the DVT recording and are therefore shown somewhat more three-dimensionally in FIG. 3b.

According to the present dislosure, said markers or anatomical structures, especially the markers 125 shown in FIG. 1 or structures of a template 115 also shown there, serve as basis for point clouds of this kind. In this way, an overall image or an overall model can be created from the first and second image data sets.

Here a coordinate transformation is determined for the point clouds 320, 325 such that the distances between the point clouds 320, 325 (or 310, 315) are minimized. In known fashion, for each point in the point cloud a closest point is determined in the other point cloud. The sum of the squares of the distances is minimized by adjusting transformation parameters, this being done iteratively until an optimal alignment is obtained between the point clouds 320, 325.

The first 3D image data mentioned are schematically represented in FIG. 3c by a three-dimensional CEREC data space 300 and the second 3D image data are schematically represented in FIG. 3d by a DVT data space 305 which is also three-dimensional. To bring the two point clouds 320 and 325 into spatial alignment, in the present example the DVT data space 305 is rotated about a first axis of rotation 330 with an angle φ and about a second axis of rotation 335 with an angle θ until alignment is secured with an empirically specified accuracy. By means of these two rotation transformations with the two angles φ and θ, the point clouds of the two data spaces 300, 305 are entirely brought into alignment as indicated by the dashed arrow lines 340, 345, 350, 355.

According to the preferred embodiment of the method according to the disclosure the following optimization problem is solved to achieve the said alignment. In this optimization, a quadratic distance minimization is in particular sought in accordance with the following relationship:

Min(dist(D1i, −D2i)) for all i,

where

−D2=R*D2+T.

In the equation R represents a rotation matrix and T a translation vector. The two terms Dli and D2i here correspond to the i data points which are contained in or available in data space 1 and data space 2. The free parameters available for the said optimization are in this case R and T. Since there are basically more data points than unknowns in this present case, an iterative approach with a least-square solution is preferably selected here for the optimization. The solution of such an approach will then correspond to the best matching transformation R|T, which describes a rigid transformation in six degrees of freedom.

If, instead of an aforementioned template 115, a natural marker located on the teeth is for example used, the number of data points i which are taken into consideration will vary. This means that outliers, for example, can be weeded out.

As an alternative a following system of equations can also be solved in which, for example, a pseudoinverse and a least-mean-square approach can be used, where:

$$R = ( \begin{matrix}{R}_{11} & {R}_{12} & {R}_{13} \\ {R}_{21} & {R}_{22} & {R}_{23} \\ {R}_{31} & {R}_{32} & {R}_{33}\end{matrix} )$$

$$and$$

$$T = ( \begin{matrix}{T}_{x} \\ {T}_{y} \\ {T}_{z}\end{matrix} )$$

With R as linearized form of an Euler angle representation the following is obtained:

$$\begin{matrix}\begin{matrix}R = {R}_{z} ( {⊖}_{z} ) \cdot {R}_{y } ( {⊖}_{y} ) \cdot {R}_{x} ( {⊖}_{x} ) \\ = [ \begin{matrix}{c}_{z} & - {s}_{z} & 0 \\ {s}_{z} & {c}_{z} & 0 \\ 0 & 0 & 1\end{matrix} ] [ \begin{matrix}1 & 0 & 0 \\ 0 & {c}_{y} & - {s}_{y} \\ 0 & {s}_{y} & {c}_{y}\end{matrix} ] [ \begin{matrix}{c}_{x} & 0 & {s}_{x} \\ 0 & 1 & 0 \\ - {s}_{x} & 0 & {c}_{x}\end{matrix} ] \\ = [ \begin{matrix}( {c}_{x} {c}_{z} - {s}_{x} {s}_{y} {s}_{z} ) & - {c}_{y} {s}_{z} & ( {s}_{x} {c}_{z} + {c}_{x} {s}_{y} {s}_{z} ) \\ ( {c}_{x} {s}_{z} + {s}_{x} {s}_{y} {c}_{z} ) & {c}_{y} {c}_{z} & ( {s}_{x} {s}_{z} - {c}_{x} {s}_{y} {c}_{z} ) \\ - {s}_{x} {c}_{y} & {s}_{y} & {c}_{x} {c}_{y}\end{matrix} ]\end{matrix} & ( 26 ) \\ {s}_{x} = sin ( {⊖}_{x} ) , \\ {c}_{x} = cos ( {⊖}_{x} ) , \\ {s}_{y} = sin ( {⊖}_{y} ) , \\ {c}_{y} = cos ( {⊖}_{y} ) , \\ {s}_{z} = sin ( {⊖}_{z} ) , \\ {c}_{z} = cos ( {⊖}_{z} ) & \end{matrix}$$

For a correspondence point i (for example, an aforementioned marker or a natural, three-dimensional point distribution)

$${M}_{i} = ( \begin{matrix}{M}_{xi} \\ {M}_{yi} \\ {M}_{zi}\end{matrix} )$$

it therefore holds that its transform satisfies the following linear transformation equation:

[symbol]

=R·Mᵢ+T

For one axis, X, for example, an overdetermined equation system of the following form is now obtained for N (N>4) markers by simple conversion:

$$( \begin{matrix} \\ \\ ⋮ \\ \end{matrix} ) = ( \begin{matrix}{M}_{x 1} & {M}_{y 1} & {M}_{y 2} & 1 \\ {R}_{11} & {R}_{12} & {R}_{13} & 1 \\ {R}_{11} & {R}_{12} & {R}_{13} & 1 \\ {R}_{11} & {R}_{12} & {R}_{13} & 1\end{matrix} ) \cdot ( \begin{matrix}{R}_{11} \\ {R}_{12} \\ {R}_{13} \\ {T}_{x}\end{matrix} )$$

In this equation system, all axes can be solved by linearization, independently of each other. The resulting linear equation system has the form.

b=Ax,

A possible solution is thus obtained by means of a pseudoinverse and a least-mean-square approach, where:

∥Ax−b∥₂^{2 min!}

or

A^{T}Ax=A^{T}b

As a result, the following balancing solution emerges:

∥A^{T}Ax−A^{T}b∥₂²min!

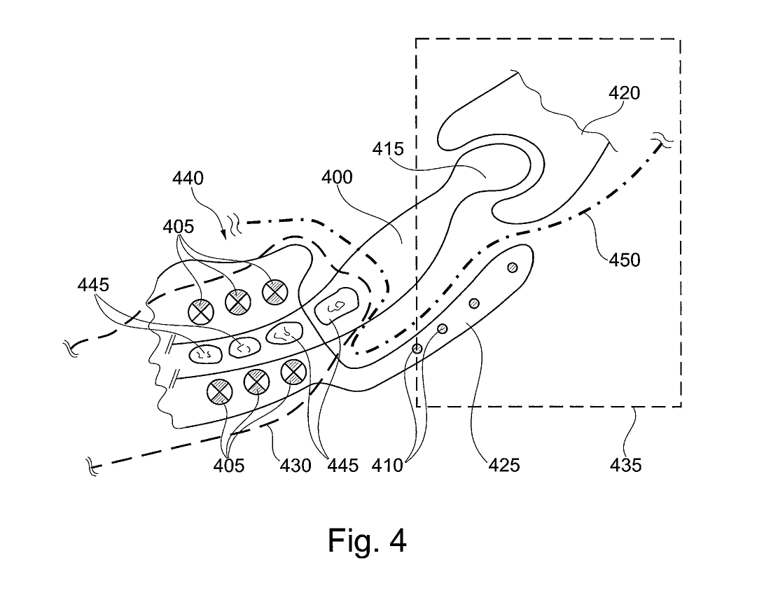

FIG. 4 shows in schematic form an exemplified top view of a ‘mandibular’ layer, that is, a layer relating to the lower jaw 400 of a patient, with the aid of which a possible image fusion using optical markers 405 and radiographic markers 410 is illustrated. It should be noted that these markers 405, 410 can also be arranged at different heights, as shown in FIG. 5 described below. The first line 430 additionally marked represents the field of view (FoV) in optical data capture by means of the said optical surface scanning (OSS) and the second line 435 also additionally marked represents the field of view in the said radiographic data capture by means of DVT. It should be emphasized that the field of view 435 in the DVT corresponds to a relatively small radiographic volume and thus radiation exposure for the patient and operating personnel is relatively low. In the present example, optical data corresponding to the first field of view 430 are captured at a number of teeth 445 which are located in the oral cavity 440 of a patient and radiographic data corresponding to the second field of view 435 are captured at one or both temporomandibular joints 415, 420 of the patient.

As can also be seen from FIG. 4 with the aid of the soft tissue-air boundary 450 marked and with the example of the radiographic markers 410, the markers 405, 410 can even be located outside the head of the patient, for example, in the vicinity of the temporomandibular joints 415, 420, on a correspondingly modified template 425.

The arrangement of the markers 405, 410 with respect to each other can be effected by means of a prescribed known spatial transformation rule for the template 425 in question. The markers 405, 410 arranged on the template 425 help to secure stability in finding a solution to the said equation system for the transformation, particularly in those situations where the said optically and radiographically acquired data overlap slightly or even not at all.

FIG. 5 shows an embodiment 500 of a template 425, already shown in FIG. 4, for enabling a described transformation. Said spherical 510, 515 or cylindrical 505 optical or radiographic markers can be implemented by means of this bite template 500. These markers 505, 510, 515, as just described, are thus preferably positioned outside the oral cavity of the patient but with a fixed spatial relationship to a particular bite block 520. Advantageously, with such a template 500 the arrangement of the balls or cylinders, such as in this case the spherical markers 515, can additionally be varied in the vertical direction in order to provide even more accurately the spatial information required for the transformation.

The method described can be realized in the form of a control program for an image generation or image display system, as is the case here, or in the form of one or more corresponding electronic control units (ECUs).

### LIST OF REFERENCE NUMBERS

100 Lower jaw layer
105, 110 Temporomandibular joints
115 Template
117 Handle
120 Rows of teeth
122, 123 Lower jaw areas
125 Holes
130, 205 CEREC scan areas
135 DVT volume
200 Fitting template
210 Generation of 3D image data (DVT)
215, 220 Identification of markers
225, 230 3D-3D registration
300 CEREC data space
305 DVT data space
310, 315 Point clouds
320, 325 Point clouds
330, 335 Axes of rotation
340-355 Arrow lines
400 Lower jaw
405 Optical markers
410 Radiographic markers
425, 500 Template
430, 435 Fields of view (FoV)
440 Oral cavity
445 Teeth
450 Soft tissue-air boundary
505-515 Markers
520 Bite block

1. Method for operating a dental diagnostic image generation system providing at least two modalities for generating three-dimensional image data, in particular for temporomandibular joint diagnosis and /or temporomandibular joint therapy, wherein for a dental object to be captured which covers at least one temporomandibular joint a first recording is generated tomographically as well as an at least second recording capturing the surface of the object to be captured, wherein the at least two-dimensional image data generated in the at least two recordings are merged by means of a multimodal registration, characterized in that the image data generation of the at least two-dimensional image data at the object to be captured is carried out with markers registered during the at least two recordings of different modalities, wherein image data generation is carried out at least by means of the tomographic first recording with a capture area limited in accordance with the markers, and that the at least two three-dimensional image data are spatially associated with respect to the temporomandibular joint by means of multimodal registration on the basis of the markers captured in the three-dimensional image data of both modalities.

2. Method as claimed in claim 1, wherein the number of markers are at least three.

3. Method as claimed in claim 1, wherein the markers are provided by a template which is positioned in the limited capture area during generation of the three-dimensional image data.

4. Method as claimed in claim 3, wherein the template has a configuration or arrangement of markers or structures which is visible in the at least two modalities.

5. Method as claimed in claim 1, wherein the multimodal registration is carried out by associating radiographic markers with artificial optical markers.

6. Method as claimed in claim 1, wherein the multimodal registration is carried out by associating natural or anatomy-based points.

7. Method as claimed in claim 1, wherein the multimodal registration is carried out with the aid of an ICP algorithm which merges the captured markers.

8. Method as claimed in claim 1, wherein the image data generation is carried out by means of a radiographic DVT process or MRT process, as well as by an optical scanning process.

9. Computer program which is set up to perform each step of a procedure in accordance with claim 1.

10. Machine-readable data medium on which the computer program according to claim 9 is stored.

11. Marker device for operating a dental diagnostic image generation system by a method in accordance with claim 1, wherein the marker device takes the form of a template which can be inserted with a perfect fit into the oral cavity of a patient.

12. Marker device as claimed in claim 11, wherein the template has a configuration or arrangement of markers or structures which is visible in the at least two modalities.

13. Dental medical image generation system which is set up to be controlled by a method in accordance with claim 1.
